(19) United States
(12) Patent Application Publication
(10) Pub. No.: US 2015/0351696 A1
(43) Pub. Date: Dec. 10, 2015

(54) BIOLOGICAL OPTICAL MEASUREMENT DEVICE AND SIGNAL SEPARATION METHOD FOR SAME

(71) Applicant: HITACHI MEDICAL CORPORATION, Tokyo (JP)

(72) Inventors: Makoto OGIHARA, Tokyo (JP); Tsukasa FUNANE, Tokyo (JP); Masashi KIGUCHI, Tokyo (JP)

(21) Appl. No.: 14/654,111

(22) PCT Filed: Dec. 12, 2013
(86) PCT No.: PCT/JP2013/083284

§ 371 Date: Jun. 19, 2015

(30) Foreign Application Priority Data
Dec. 27, 2012 (JP) 2012-284994

(51) Int. Cl. A61B 5/00; A61B 5/1455; A61B 5/145; A61B 5/026

(52) U.S. Cl. CPC A61B 5/7203; A61B 5/0261; A61B 5/742; A61B 5/14552; A61B 5/14546; A61B 5/14553; A61B 2562/0238; A61B 2562/0242

(57) ABSTRACT

The present invention comprises an analysis unit for applying a plurality of candidate delay times to either data with a short SD distance or data with a long SD distance acquired in advance and calculating a separation degree indicating the degree of separation of the skin blood flow signal after separation using the TDD-ICA method for each candidate delay time; a delay time determination unit for determining the optimal delay time according to the separation degree; and a display data generation unit for generating display data to display a waveform before the separation and a waveform of the cerebral blood flow signal as well as a waveform of the skin blood flow signal after the separation on a display unit so as to be compared with each other when displaying the result of the separation using the determined delay time.

### TECHNICAL FIELD

The present invention relates to a biological optical measurement technique using visible light or near-infrared light, and in particular, to a technique for separating and eliminating the influence of surface layer components such as skin blood flow components mixed with signal components.

### BACKGROUND ART

In the brain function measurement using a biological optical measurement device, the separation of a deep blood flow component (cerebral blood flow signal) mainly including a signal from the brain and a shallow blood flow component (skin blood flow signal) mainly including a signal from the skin, in the blood flow information, is effective. For the separation of the deep blood flow component and shallow blood flow component signals, there is a technique called an independent component analysis (ICA) method. In the ICA method, the shallow blood flow component is removed using the data measured at a plurality of light receiving positions.

In addition, there is also a method of removing the shallow blood flow component by performing data processing based on the ICA method using a plurality of pieces of data (measurement data with a short SD distance and measurement data with a long SD distance) measured at a distance (Source Detector distance: SD distance) between the irradiation point and the light receiving point that are different (for example, refer to PTL 1).

In addition, it is known that it is possible to extract the independent component with high accuracy by using a time delayed decorrelation (TDD)-ICA method, in which a calculation is performed by applying a delay time to data, when applying the ICA method (for example, refer to NPL 1).

### CITATION LIST

### Patent Literature

[PTL 1] Pamphlet of International Publication No. WO2012/005303

### Non Patent Literature

[NPL 1] A. Ziehe and K.-R. Muller: “TDSEP—an efficient algorithm for blind separation using time structure”, In Proceedings of 1998 International Conference on Artificial Neural Networks (ICANN' 98), 1988, pp. 675-680

### SUMMARY OF INVENTION

### Technical Problem

For the delay time used in the TDD-ICA method, since the timing of changes in the blood flow component differs depending on the length of the task or the object, the optimal delay time differs for each object and each task.

However, even for an experienced user, setting an appropriate delay time for each measurement requires trial and error, and man-hours until the appropriate delay time can be set is a burden. It is difficult for a less experienced user to set the delay time. Even if the delay time is set, the user cannot grasp whether or not the skin blood flow signal can be appropriately separated by the set delay time.

The present invention has been made in view of the above situation, and it is an object of the present invention to provide a technique by which it is possible to grasp the separation of the skin blood flow signal and the cerebral blood flow signal without increasing the burden on the user when separating the signal components of a deep layer portion and a surface layer portion in the biological optical measurement.

### Solution to Problem

The present invention applies a plurality of candidate delay times to at least one of a plurality of principal components obtained by applying the principal component analysis to data measured in the arrangement of short SD distance and data measured in the arrangement of long SD distance that have been acquired in advance. In addition, for each candidate delay time applied, the skin blood flow signal and the cerebral blood flow signal are separated from each other. Then, using a plurality of pieces of data (measurement data with a short SD distance and measurement data with a long SD distance) measured at different SD distances, data processing based on the ICA method is performed for separation into the skin blood flow signal and the cerebral blood flow signal. In addition, a separation degree indicating the degree of separation of the skin blood flow signal is calculated, and the optimal delay time is determined according to the separation degree. When displaying a result of the separation using the determined delay time, waveforms in which a cerebral blood flow signal and a skin blood flow signal before the separation are mixed, a waveform based on a cerebral blood flow signal and a waveform based on a skin blood flow signal after the separation are displayed so as to be compared with each other.

### Advantageous Effects of Invention

According to the present invention, simply by displaying the waveforms in which the cerebral blood flow signal and the skin blood flow signal before the separation are mixed, the waveform based on the cerebral blood flow signal and the waveform based on the skin blood flow signal after the separation so as to be compared with each other, the user can check whether or not the waveform based on the cerebral blood flow signal after the separation and the waveform based on the skin blood flow signal after the separation are separated from each other. Therefore, when separating the signal components of the deep layer portion and the surface layer portion in the biological optical measurement, it is possible to check the separation of the skin blood flow signal and the cerebral blood flow signal without increasing the burden on the user.

### BRIEF DESCRIPTION OF DRAWINGS

FIG. 1 is a block diagram showing the overall biological optical measurement device of an embodiment of the present invention.

FIG. 2 is a diagram for explaining an example of the probe arrangement of the embodiment of the present invention.

FIG. 3 is a functional block diagram of a data processing unit 400 of the embodiment of the present invention.

FIG. 4 is a diagram for explaining the principle of separating a deep portion component and a shallow portion component from each other from the reception result at two long and short SD distances.

FIG. 5 is a diagram for explaining a separation method based on the TDD-ICA method using the delay time.

FIG. 6 is a diagram for explaining a separation degree display screen of the embodiment of the present invention.

FIG. 7 is a diagram for explaining a result display screen of the embodiment of the present invention.

FIG. 8 is a flowchart of the delay time determination process of the embodiment of the present invention.

### DESCRIPTION OF EMBODIMENTS

Hereinafter, an embodiment to which the present invention is applied will be described. In addition, in all diagrams for explaining the embodiment of the invention, the same reference numerals are given to component having the same functions unless otherwise stated, and repeated explanation thereof will be omitted.

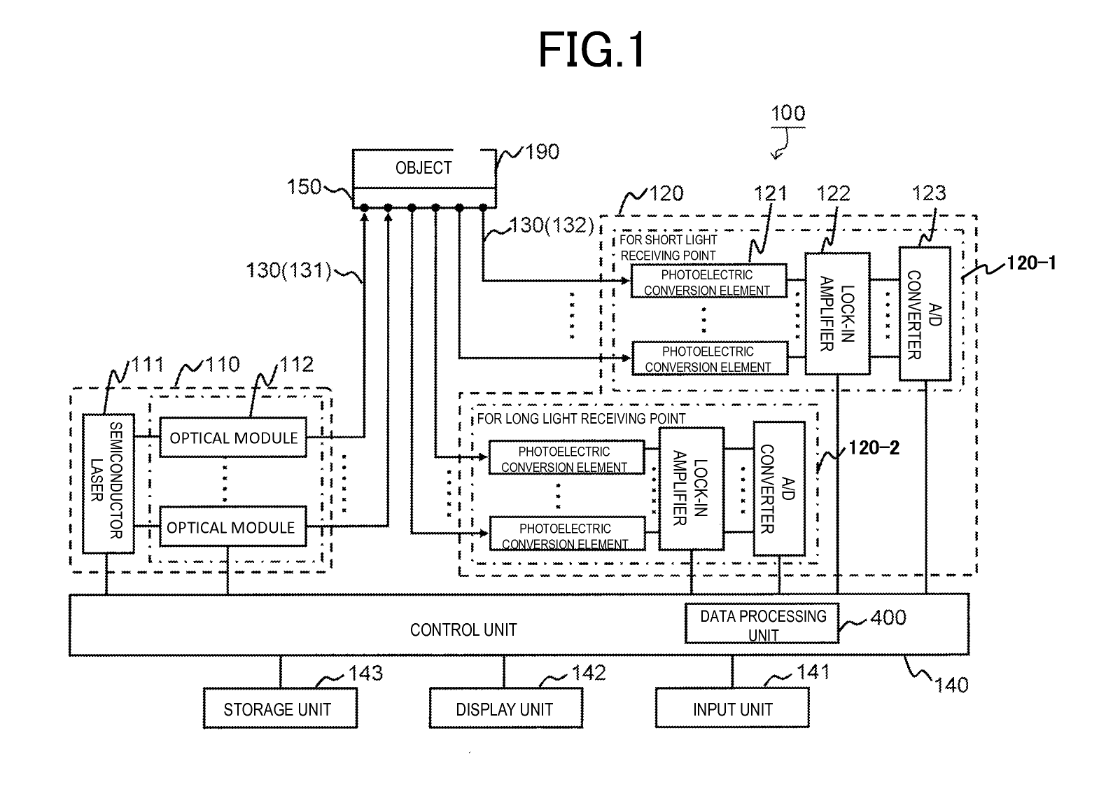

First, a biological optical measurement device of the present embodiment will be described. The biological optical measurement device is a device that emits near-infrared light to the inside of the body, detects light that is reflected from the vicinity of the surface of the body or is transmitted through the body (hereinafter, simply referred to as transmitted light), and generates an electrical signal corresponding to the intensity of the light. As shown in FIG. 1, a biological optical measurement device 100 includes alight source unit 110 that emits near-infrared light, a light receiving unit 120 that measures transmitted light and converts the transmitted light into an electrical signal, and a control unit 140 that controls the driving of the light source unit 110 and the light receiving unit 120 and processes the data output from the light receiving unit.

The light source unit 110 emits light to an irradiation point set in advance on an object 190. The light source unit 110 includes a semiconductor laser 111 that emits light having a predetermined wavelength and a plurality of optical modules 112 including modulators that modulate light emitted from the semiconductor laser 111 at different frequencies. The optical modules 112 are provided, for example, as many as the irradiation points. Output light from each of the optical modules 112 is emitted to a predetermined measurement region of the object 190 through an optical fiber 130 (irradiation light optical fiber 131).

Irradiation is performed through each probe of a probe holder 150 attached to the object 190, and light is emitted to predetermined regions of the object 190 from a plurality of irradiation points set in advance. The optical fiber 130 is fixed to the probe holder 150. For example, the probe holder 150 is fixed to the head of the object 190.

It is assumed that the wavelength of the light output from the light source unit 110 depends on the spectral characteristics of a target substance in the body. For example, when measuring the oxygen saturation or the blood volume from the concentration of hemoglobin (Hb) and oxygenated hemoglobin (HbO₂) , one or more wavelengths are selected from light in the wavelength range of 600 nm to 1400 nm and are used.

For example, when measurement targets are two kinds of oxygenated hemoglobin (HbO₂: oxyHb) and deoxygenated hemoglobin (deoxyHb), the light source unit 110 is configured to generate light beams having two wavelengths, for example, 780 nm and 830 nm, corresponding to the two kinds of measurement targets. The light beams having two wavelengths are mixed at the irradiation point, and are emitted from one light irradiation position of the probe holder 150.

The light receiving unit 120 receives transmitted light that has been guided through the optical fiber 130 (light receiving optical fiber 132) from a plurality of measurement points in the measurement region, and outputs the transmitted light to the control unit 140 as digital data. In the present embodiment, light emitted to the irradiation point and propagating through the object 190 is received at a plurality of light receiving points set in advance on the object 190, and is output as measurement data. Light reception is performed at a light receiving point corresponding to each irradiation point on the probe holder 150.

In the present embodiment, measurement data is divided into a cerebral blood flow (deep portion component) and a skin blood flow (shallow portion component) using the TDD-ICA method as will be described later. In the TDD-ICA method, light emitted from one irradiation point is received at least two light receiving points whose distances (Source Detector distances: SD distances) from the irradiation point are different. Although the case in which light is received at two light receiving points having different SD distances is described in the present embodiment, a configuration may be adopted in which a plurality of light receiving points and a plurality of irradiation points are present and a plurality of pieces of measurement data having different SD distances are obtained.

Hereinafter, the light receiving point whose distance from the irradiation point is long is referred to as a long light receiving point, and the light receiving point whose distance from the irradiation point is shorter than of the long light receiving point is referred to as a short light receiving point. In addition, measurement data obtained from the signal received at the short light receiving point is referred to as short measurement data, and measurement data obtained from the signal received at the long light receiving point is referred to as long measurement data.

An example of the arrangement of probes 151, which are connected to the light source unit 110 and the light receiving unit 120, in the probe holder 150 to realize a plurality of above SD distances, will be described. FIG. 2 is a diagram for explaining the arrangement example.

In the present embodiment, in order to separate the deep blood flow component and the shallow blood flow component from each other, an irradiation point 151ap, a short light receiving point 151bp, and a long light receiving point 151cp are disposed so that measurement according to a plurality of SD distances is realized and light to be received propagates through both gray matter and a scalp. Specifically, as shown in FIG. 2, the probes 151 are disposed in a grid. In the diagram, ▴ indicates a probe 151a connected to the irradiation light optical fiber 131 connected to the light source unit 110, and indicates the above-described irradiation point 151ap. In addition, in the diagram, ▪ and indicate probes 151b and 151c connected to the light receiving optical fiber 132 connected to the light receiving unit 120, and indicate the above-described short light receiving point 151bp and long light receiving point 151cp, respectively.

The point between the irradiation point 151ap and each of the short light receiving point 151bp and the long light receiving point 151cp on the probe holder 150 is referred to as a measurement point. In addition, light is received at two light receiving points (short light receiving point 151bp and long light receiving point 151cp) for each irradiation point 151ap, and a deep portion component and a shallow portion component are obtained from the result. In the present embodiment, a set of irradiation point 151ap, short light receiving point 151bp, and long light receiving point 151cp to obtain a set of deep portion component and shallow portion component is referred to as a measurement channel.

In the present embodiment, as shown in FIG. 1, the light receiving unit 120 includes the light receiving unit 120 for long light receiving points and the light receiving unit 120 for short light receiving points for each measurement channel.

In order to obtain the measurement data from the signals received at the short light receiving point 151bp and the long light receiving point 151cp, each light receiving unit 120 of the present embodiment includes: a photoelectric conversion element 121 such as a photodiode that converts the received light into the amount of electricity corresponding to the amount of light; a lock-in amplifier 122 that receives the electrical signal from the photoelectric conversion element 121 and selectively detects a modulation signal corresponding to the light irradiation position; and an A/D converter 123 that converts the output signal of the lock-in amplifier 123 into a digital signal.

The photoelectric conversion element 121 is provided as many as the long light receiving points and the short light receiving points. The lock-in amplifier 122 selectively detects a modulation signal corresponding to the two wavelengths for each irradiation point 151ap of the probe holder 150. The detected modulation signal indicates a change in the amount of hemoglobin.

Therefore, the modulation signal is referred to as a hemoglobin amount change signal. That is, each of short measurement data and long measurement data that are output is a hemoglobin amount change signal. The “amount of hemoglobin” includes the “amount of oxygenated hemoglobin” and the “amount of deoxygenated hemoglobin”.

As shown in FIG. 1, the control unit 140 includes a data processing unit 400 that performs processing on the short measurement data and the long measurement data output from the light receiving unit 120. The data processing unit 400 generates display data by processing the short measurement data and the long measurement data received from the light receiving unit 120.

When the short measurement data and the long measurement data are the hemoglobin amount change signal, display data to be generated is, for example, a graph showing an oxygenated hemoglobin (oxyHb) concentration change, a deoxygenated hemoglobin (deoxyHb) concentration change, and a total hemoglobin (TotalHb) concentration change of each of the cerebral blood flow component and the skin blood flow component for each measurement channel or an image obtained by plotting these on the two-dimensional image of the object 190.

In the present embodiment, the data processing unit 400 extracts separated components (TDD-ICA processing) by performing independent component analysis (ICA) after applying a predetermined delay time T to at least one of the long measurement data and the short measurement data, and reconstructs the extracted separated components for separation into a deep portion component and a shallow portion component.

Therefore, as shown in FIG. 3, the data processing unit 400 of the present embodiment includes an analysis section 410 that performs TDD-ICA processing, a delay time determination section 420 that determines an optimal delay time to be used this time, and a display data generation section 430 that generates display data from a deep portion component and a shallow portion component obtained by applying the optimal delay time.

In addition, as shown in FIG. 1, a display unit 142 for displaying the processing result of the data processing unit 400, for example, a generated image, a storage unit 143 for storing data required for the processing of the data processing unit 400 or the processing result, and an input unit 141 for inputting various commands required for the operation of the biological optical measurement device 100 are connected to the control unit 140 of the present embodiment.

Each function of the control unit 140 is realized when a CPU provided in the control unit 140 loads a program stored in the storage unit 143 in advance to a memory and executes the program.

Each function of the control unit 140 may also be realized by hardware, such as a programmable logic device (PLD).

Prior to explaining the TDD-ICA processing performed by the analysis section 410, the principle of separation into a deep portion component and a shallow portion component of the brain from the result received at two SD distances will be described. FIG. 4 shows an example of the measurement cross-section in the case of receiving light at two SD distances. The short light receiving point 151bp and the long light receiving point 151cp are disposed at the SD distance of 15 mm (short SD distance) and the SD distance of 30 mm (long SD distance), respectively. Light 300 emitted through the irradiation point 151ap from the light source unit 110 is incident from the scalp and propagates in all directions in tissue. The light 300 (301) received at the short light receiving point 151bp is transmitted through a shallow portion on the average compared with the light 300 (302) received at the long light receiving point 151cp.

Since the average optical path length in tissue is different, the partial average optical path length in each layer of the head is changed. It is known that, if the SD distance is approximately 10 mm or more and 40 mm or less, the dependence of the partial optical path length of the scalp on the SD distance is small but the gray matter has an almost linear dependence on the SD distance.

In a near-infrared spectroscopy (NIRS) using near-infrared light as light to be transmitted, the NIRS signal strength is proportional to the partial optical path length of a part where a blood flow change occurs if the blood flow change is the same. Accordingly, it is expected that, as the SD distance increases, a component from the brain (cerebral blood flow) in the NIRS measurement signal increases but there is no change in a component from the skin (skin blood flow).

That is, in the short measurement data, the brain is not included in the light propagation path as much as the long measurement data. Accordingly, the relationship of the short measurement data, the long measurement data, the cerebral blood flow, and the skin blood flow is expressed by the following Expressions (1) and (2).

long measurement data=skin blood flow+cerebral blood flow (large) (1)

short measurement data=skin blood flow+cerebral blood flow (small) (2)

Therefore, there is also a method of obtaining the cerebral blood flow by subtracting the short measurement data from the long measurement data from Expressions (1) and (2).

In the ICA method, the skin blood flow and the cerebral blood flow are extracted, as independent components, from two or more pieces of measurement data. The ICA method is a method of extracting a plurality of independent components from signals as results of light reception at a plurality of measurement points and separating these into a component from the brain or a component from the skin, and is an analysis method capable of separating the linearly mixed signals without priori information. There are many signal sources, and this is effective for the analysis of data measured at multiple points.

In this case, the SD distances of a plurality of pieces of measurement data to be acquired may be equal. However, in the case of a method in which both the long SD data and the short SD data are included in the measurement data, more accurate separation becomes possible by performing a contribution rate calculation using the method disclosed in PTL 1.

The TDD-ICA processing performed by the analysis section 410 is for increasing the accuracy by performing a process by applying a delay time to at least one piece of data using a plurality of pieces of data measured at two different SD distances in the ICA method for separating the deep portion component and the shallow portion component from each other.

In the TDD-ICA processing, in order to extract independent components, principal components are repeatedly rotated so that all of second-order correlations between the signals in consideration of a plurality of time differences become zero. Setting all of the second-order correlations to zero is simultaneously diagonalizing a plurality of covariance matrices corresponding to the respective time differences. A plurality of time differences are obtained by dividing the applied delay time T by a number set in advance.

FIG. 5 is a diagram for explaining the TDD-ICA processing performed by the analysis section 410 of the present embodiment. First, the analysis section 410 performs principal component analysis (PCA) on short measurement data 512 and long measurement data 511, thereby separating the data into principal components C₁ and C₂.

Then, the amount of delay of 1 or more is applied to at least one of the principal components C₁ and C₂. The applied amount of delay is expressed as k×dt (k=0, 1, 2, . . . , K) using a unit delay time dt obtained by dividing the applied delay time T by K (K is an integer of 1 or more). The delay time T is stored in the storage unit 143 in advance. Then, the principal components C₁ and C₂ to which the amount of delay has been applied are simultaneously diagonalized by applying a correlation matrix based on the independent component analysis, thereby performing separating into two components. Data analysis of the operation is performed, and two separated components obtained herein by the data analysis are referred to as a first separated component 521 and a second separated component 522.

Although there is a detailed description in PTL 1, the analysis section 410 performs reconstruction processing on the first separated component 521 and the second separated component 522 after the data analysis, thereby performing separation into a deep blood flow waveform (deep portion component) 531 and a shallow blood flow waveform (shallow portion component) 532. In the reconstruction processing, the deep contribution rate of the first separated component 521 and the second separated component 522 after the separation is calculated. The deep contribution rate is a value indicating the rate of each of the separated components 521 and 522 contributing to the deep blood flow. Using the deep contribution rate, the analysis section 410 calculates the deep blood flow waveform 531, and calculates the shallow blood flow waveform 532 by subtracting the deep blood flow waveform 531 from the measured waveform (all components).

In addition, the analysis section 410 performs the above-described TDD-ICA processing on the long measurement data 511 and the short measurement data 512 for each measurement channel.

The delay time determination section 420 determines an optimal delay time Tbest, which is applied to at least either the long measurement data or the short measurement data that has been obtained, as described above. In the present embodiment, the delay time determination section 420 prepares a plurality of candidate delay times T in advance, and the analysis section 410 calculates the deep portion component 531 and the shallow portion component 532 when each delay time is applied. Then, using the result, a separation degree is calculated as an index indicating the degree of separation of the deep portion component 531 and the shallow portion component 532. Then, the optimal delay time Tbest is determined among the candidate delay times T. In the present embodiment, the candidate delay time T by which the separation degree is maximized is determined as the optimal delay time Tbest.

The separation degree is an indicator to determine the good or bad of the processing result. In the present embodiment, for example, a broad spectrum coefficient is used as the separation degree. The broad spectrum coefficient is defined as a value obtained by dividing the average value of the root mean square (RMS) of hemoglobin changes for all channels by the standard deviation, for example. The separation degree is calculated as a difference between the broad spectrum coefficients of the deep portion component and the shallow portion component. This is to use the fact that the skin blood flow indicated by the shallow blood flow waveform is overall the same movement and accordingly the broad spectrum is high and that the cerebral blood flow indicated by the deep blood flow waveform is partially active and accordingly the broad spectrum is low.

In addition, the separation degree may be calculated from the deep portion component and the shallow portion component of oxygenated hemoglobin (oxyHb), or may be calculated from the deep portion component and the shallow portion component of deoxygenated hemoglobin (deoxyHb), or maybe calculated from the deep portion component and the shallow portion component of both of the oxygenated hemoglobin (oxyHb) and the deoxygenated hemoglobin (deoxyHb). The calculation method may be selected. In addition, the separation degree may be calculated from the correlation with a shallow portion component or the like using other biological signals, for example, measurement results of a laser Doppler flowmeter (LDF).

When the task is repeatedly performed with the object, the separation degree may be calculated using the task synchronization with a deep portion signal or a shallow portion signal. In this case, for example, in the deep portion signal, time-series data is divided for each trial, and the average of the correlation coefficients in all combinations to select two trials from the number of trials is calculated as task synchronization. This is particularly effective for obtaining the deep portion signal with high task reproducibility.

In addition, the separation degree may also be calculated using the correlation between the oxygenated hemoglobin change and the deoxygenated hemoglobin change (oxyHb, deoxyHb) in the deep portion signal or the shallow portion signal. In this case, for example, a difference in correlation coefficient between the oxygenated hemoglobin change and the deoxygenated hemoglobin change (oxyHb, deoxyHb) in the deep portion signal and the shallow portion signal may be calculated as the separation degree. A case indicating a positive correlation between the oxygenated hemoglobin change and the deoxygenated hemoglobin change is particularly effective for separating the systemic signal that has been reported.

In addition, the separation degree may also be calculated from the correlation coefficient between a waveform estimated from the hemodynamic response function and the deep portion signal or the shallow portion signal. In this case, for example, a brain activity waveform is estimated from the convolution of the hemodynamic response function and the rectangular wave of the period.

The user may select one of the above separation degree calculation methods, or may perform an analysis using a plurality of calculation methods simultaneously.

The delay time determination section 420 stores the calculated separation degree in the storage unit 143 so as to match the candidate delay time T. The calculation result may be presented to the user. That is, the calculation result may be displayed on the display unit 142. FIG. 6 shows an example of the display screen (separation degree display screen 600) in this case.

As shown in FIG. 6, the separation degree display screen 600 includes a candidate delay time display region 601 and a separation degree display region 602 to display the separation degree of each candidate delay time T. Here, a case where the candidate delay time set in advance is 3, 5, 10, 20, and 30 seconds is shown as an example. In the separation degree display region 602, only the separation degree regarding the oxygenated hemoglobin concentration change (oxyHb) may be displayed, or the separation degree of each of the deoxygenated hemoglobin concentration change (deoxyHb) and the total hemoglobin concentration change (Total) may be further displayed. In addition, a display form of a set of the separation degree and the candidate delay time showing the best separation degree (maximum value) maybe changed and displayed so as to be distinguishable from others.

The display data generation section 430 generates display data to be displayed on the display unit 142, from the deep portion component 531 and the shallow portion component 532 obtained by the analysis section 410, by applying the optimal delay time Tbest determined by the delay time determination section 420. The display data is for displaying the waveforms, in which the cerebral blood flow signal and the skin blood flow signal before the separation are mixed, the waveform based on the cerebral blood flow signal and the waveform based on the skin blood flow signal after the separation, so as to be compared with each other, and includes the optimal delay time Tbest and at least one of the wavelengths of the components before the separation, the deep portion component 531, and the shallow portion component 532. In addition, the display data may further include a percentage of the deep portion component 531 with respect to a total. The waveform of the component before the separation is generated from the long measurement data 511.

In this case, an example of a result display screen 700 that is generated by the display data generation section 430 and is displayed on the display unit 142 is shown in FIG. 7.

As shown in FIG. 7, the result display screen 700 includes a waveform display field 701 for displaying the waveform of each component, a percentage display field 702 for displaying the percentage of the deep portion component to a total, and an information display field 703 for displaying the optimal delay time Tbest.

In FIG. 7, in the waveform display field 701, a waveform of the deep portion component (deep blood flow waveform 711), a waveform of the shallow portion component (shallow blood flow waveform 712), and a waveform obtained from long measurement data (total blood volume 713) that is a waveform before separation are displayed for each measurement channel. That is, display data is generated for each of the deep portion component and the shallow portion component obtained from the short measurement data and the long measurement data that have been received in sets of a plurality of irradiation points, a plurality of short light receiving points, and a plurality of long light receiving points, and is displayed in the waveform display field 701. The waveform display field 701 may be disposed so as to be map-displayed in accordance with the position of the long measurement data measuring point or the short measurement data measuring point.

In the percentage display field 702, for each of oxygenated hemoglobin (oxyHb) and deoxygenated hemoglobin (deoxyHb), the percentage of the deep portion component is displayed for each measurement channel. Display may be for either of oxygenated hemoglobin (oxyHb) and deoxygenated hemoglobin (deoxyHb). It is also possible to adopt a configuration in which an object to be displayed is selectable.

A candidate delay time adopted as the optimal delay time and the separation degree are displayed in the information display field 703.

Next, the flow of the delay time determination process of the delay time determination section 420 will be described. FIG. 8 is a process flow of the delay time determination process of the present embodiment. The delay time determination section 420 applies a candidate delay time set in advance to at least one of a plurality of principal components obtained by applying the principal component analysis for the short measurement data and the long measurement, causes the analysis section to perform separate into a deep portion component and a shallow portion component, repeats the calculation of the separation degree, which is an index indicating the degree of separation, while changing the candidate delay time, and sets the candidate delay time, which is a best indicator, as an optimal candidate delay time.

This process is started after acquiring the short measurement data and the long measurement data for all measurement channels. Here, an example will be described in which M (M is an integer of 1 or more) candidate delay times are applied. i is a counter that counts the candidate delay time, and T[i] is the i-th candidate delay time. In addition, the number of measurement channels is set to N (N is an integer of 1 or more). j is a counter that counts the number of measurement channels.

The delay time determination section 420 initializes (i=1, j=1) a counter (step S1001). Then, the delay time determination section 420 sets an i-th candidate delay time T[i] (step S1002).

The delay time determination section 420 causes the analysis section 410 to perform the TDD-ICA processing for each measurement channel j using the candidate delay time T[i]. First, the analysis section 410 performs TDD-ICA processing on short measurement data SSD[j] and long measurement data LSD[j] acquired in the measurement channel j (step S1003), thereby obtaining a deep portion component DEP[i, j] and a shallow portion component SHA[i, j] for the measurement channel j. In the present embodiment, the deep portion component DEP [i, j] and the shallow portion component SHA[i, j] of each of the amount of oxygenated hemoglobin, the amount of deoxygenated hemoglobin, and the total amount of hemoglobin are obtained. The analysis section 410 stores the obtained deep portion component DEP[i, j] and shallow portion component SHA[i, j] in the storage unit 143 so as to match the candidate delay time T[i] and the measurement channel j (step S1004). The delay time determination section 420 repeats the processing of steps S1003 and 51004 for the data of all measurement channels (steps S1005 and S1006).

The delay time determination section 420 calculates a separation degree SEP [i] based on the candidate delay time T[i] using the deep portion component and the shallow portion component for all measurement channels of the candidate delay time T[i] (step S1007). As the separation degree, for example, the broad spectrum coefficient described above is calculated. The delay time determination section stores the calculated separation degree SEP [i] in the storage unit 143 so as to match the candidate delay time T[i]. The delay time determination section 420 repeats the above processing for all candidate delay times (steps S1008 and S1009).

The delay time determination section 420 determines the optimal delay time Tbest based on the separation degree of each of the obtained candidate delay times (step S1010). When the broad spectrum coefficient is used as the separation degree, a candidate delay time by which the separation degree is maximized is selected as the optimal delay time.

The display data generation section 430 reads a deep portion component DEP [Tbest, j] and a shallow portion component SHA[Tbest, j] that are stored in the storage unit 143 so as to match the optimal delay time Tbest, generates the result display screen 700 as display data, and displays the result display screen 700 on the display unit 142 (step S1011).

Although the delay time determination section 420 performs separation processing for each measurement channel in the delay time determination process described above, the present invention is not limited thereto. The separation processing may also be performed in parallel for all measurement channels.

In addition, although one of the candidate delay times set in advance that corresponds to the best separation degree is set as the optimal delay time in the embodiment described above, the determination of the optimal delay time is not limited thereto. For example, it may also be possible to determine the candidate delay time Tbest by which the separation degree is maximized and then to perform a search in the vicinity of the best candidate delay time Tbest and set the delay time corresponding to the best separation degree as an optimal delay time.

In the search, for example, as the vicinity of the best candidate delay time Tbest, a plurality of times in a predetermined range having the candidate delay time Tbest at the center are set as new candidate delay times. Then, in the same manner as described above, the TDD-ICA analysis is performed for each candidate delay time, and the separation degree is calculated from the result. The TDD-ICA analysis is performed by the analysis section 410, and the calculation of the separation degree and the determination of the optimal delay time are performed by the delay time determination section 420.

In this manner, it is possible to set the best delay time as the optimal delay time without being limited to the candidate delay time set in advance.

In the present embodiment, the optimal delay time Tbest is determined using the separation degree as an indicator of the separation of two different signals. However, the present invention is not limited thereto. For example, it may also be possible to use a mixing degree indicating the degree of mixing of two signals, which is opposite to the separation, as an indicator of the separation. If the mixing degree is defined as the inverse of the separation degree, the delay time determination section 420 selects a candidate delay time, by which the mixing degree is minimized, as the optimal delay time Tbest using the broad spectrum coefficient described above.

The candidate delay time may not be set in advance. For example, time after every predetermined time interval Δt from 0 (sec) may be set as the candidate delay time. In this case, it is assumed that the maximum candidate delay time does not exceed the total measurement time S (sec) to acquire the short measurement data and the long measurement data of the present embodiment.

In addition, the optimal delay time is influenced by the total measurement time S or a stimulation period. Therefore, the optimal delay time may be calculated in advance based on the total measurement time S or the stimulation period of the measured data, and may be set as the candidate delay time. In this case, for example, a half of the total measurement time S or the stimulation period may be set to the candidate delay time.

In the present embodiment, data to apply the candidate delay time, that is, data to be analyzed in FIG. 5, is a plurality of principal components in the principal component analysis. However, the present invention is not limited thereto. The original measurement data may also be data obtained by a method using a signal separation technique, such as factor analysis, multiple regression analysis, and cluster analysis.

The terms “cerebral blood flow component” and “skin blood flow component” used herein are for the convenience of designation, and are independent components that are formally separated by the gradient of the weighting value for the SD distance by the above method and NIRS signals reconstructed by the plurality of separated independent components. Therefore, for example, a possibility that not only the biological signal of deep tissue including the brain but also a fluctuation component of blood in the blood vessels in the skull is included in the “cerebral blood flow component” may also be considered. In addition, not only the biological signal of shallow tissue but also components from parts other than the brain, that is, a systemic biological signal, device noise, noise due to body movement, and the like may be included in the “skin blood flow component”.

In the present embodiment, the delay time determination section automatically determines a candidate delay time corresponding to the best separation degree, among the candidate delay times set in advance, as the optimal delay time. However, the determination of the optimal delay time is not limited thereto. For example, the user may designate the optimal delay time while viewing the separation degree of each candidate delay time displayed on the separation degree display screen 600. In this case, the separation degree display screen 600 includes a designation receiving button to receive the designation of the optimal delay time.

In addition, the user may be able to enter a new candidate delay time. In this case, as shown in FIG. 6, the separation degree display screen 600 further includes a candidate delay time input region 603 for receiving the input of the candidate delay time and an input delay time separation degree display region 604 for displaying the separation degree based on the input candidate delay time. The delay time determination section 420 calculates the separation degree for the newly input candidate delay time, and displays the separation degree in the input delay time separation degree display region 604. Also in this case, the separation degree is calculated using the result obtained after the analysis section 410 calculates the deep portion component and the shallow portion component using the short measurement data and the long measurement data for each channel.

In this case, the delay time determination section 420 may calculate and display the separation degree whenever the user enters a new value in the candidate delay time input region 603. In this case, a determination button 605 to receive the intention of the optimal delay time determination by the user may be provided. The delay time determination section 420 sets the candidate delay time input to the candidate delay time input region 603 as the optimal delay time when the user receives the pressing of the determination button 605.

The separation degree display screen 600 may include a reception field 606 to receive the instruction regarding whether to automatically determine (Auto Set) or manually determine (Manual Set) the optimal delay time. The automatic determination (Auto Set) is a method in which the delay time determination section 420 automatically determines the optimal delay time among the candidate delay times set in advance, and the manual determination (Manual Set) is a method of determining the optimal delay time among the candidate delay times input by the user.

The separation degree display screen 600 may include a START button 607 for receiving an instruction to start the delay time determination process and a progress bar 608 showing the progress of the delay time determination process.

In the method disclosed in PTL 1, the delay time is uniquely set. However, since the optimal delay time changes with the length of the measurement data acquisition time, task, or the like, it is difficult to check whether or not the set delay time is the best delay time. For this reason, it has been difficult to search for the optimal value.

As described above, according to the present embodiment, the optimal delay time is determined among a plurality of candidate delay times set in advance. In this manner, it is possible to reduce the user's setting burden. In addition, the separation degree for each candidate delay time is calculated, and the optimal candidate delay time is automatically determined. In this manner, the user's setting burden is further reduced. In this case, the accuracy of the separation is also increased by setting the candidate delay time, by which the separation degree is maximized, as the optimal delay time. In addition, the accuracy of the separation is further increased by performing a search in the vicinity of the candidate delay time by which the separation degree is maximized and setting the delay time corresponding to the higher separation degree as the optimal delay time. In addition, according to the present embodiment, since the separation degree of each candidate delay time is displayed, the user can easily grasp the separation degree for each delay time. Since the user can set the candidate delay time and the separation degree of the set candidate delay time is presented to the user, the user can set the delay time to obtain the desired separation degree. The degree of freedom of delay time setting by the user is increased.

That is, according to the present embodiment, since the optimal delay time can be easily set, it is possible to reduce the user's setting burden. The optimal delay time can be determined without increasing the burden on the user, and the accuracy of the separation is increased since the separating processing is performed using the optimal delay time.

In addition, the display of the result is displayed so as to be able to compare the deep portion component, the shallow portion component, and all components for each measurement channel. Therefore, the user can easily grasp how many deep portion components or shallow portion components are included in the original waveform. In this case, the separation degree that is an index indicating the degree of separation is also displayed. Therefore, according to the present embodiment, for the used delay time, separation degree, and wavelength after separation, good or bad of the analysis is clearly expressed so as to be easily understood by the user.

According to the present embodiment, a plurality of candidate delay times are applied to either the short SD-distance data or the long SD-distance data acquired in advance. In addition, there are provided: the analysis section 410 that calculates a separation degree indicating the degree of separation of the skin blood flow signal after separation using the TDD-ICA method for each applied candidate delay time; the delay time determination section 420 that determines the optimal delay time according to the separation degree; and the display data generation section 430 that generates display data for displaying the waveform before the separation and the waveform of the cerebral blood flow signal and the waveform of the skin blood flow signal after the separation on the display unit 142 so as to be compared with each other when displaying the result of the separation using the determined delay time.

Specifically, there are provided: one or more light source units 110 that emit light to the irradiation point set in advance on the object 190; one or more pairs of light receiving units 120 that receive light emitted to the irradiation point and propagating through the object 190 at a light receiving point set in advance on the object 190, that are disposed such that the distance between the irradiation point and the light receiving point for measuring the short measurement data is shorter than the distance between the irradiation point and the light receiving point for measuring the long measurement data, and that output at least one of the short measurement data and the long measurement data obtained from the received signal; the data processing unit 400 that performs processing on the short measurement data and the long measurement data; and the display unit 142 that displays the short measurement data and the long measurement data processed by the data processing unit 400 and analysis data of the short measurement data and the long measurement data. The data processing unit 400 includes: the analysis section 410 that extracts separated components by performing independent component analysis including a process of applying a delay time to at least one of a plurality of principal components obtained by an operation using at least one of the short measurement data and the long measurement data and separates the separated components into a deep portion component and a shallow portion component by reconstructing the separated components; the delay time determination section 420 that determines a delay time from the candidate delay times indicated in the process of applying the delay time; and the display data generation section 430 that generates display data displayed on the display unit from the deep portion component and the shallow portion component obtained by applying the delay time.

Therefore, the user can easily check the result, and can grasp the result numerically. Thus, according to the present embodiment, in a method of separating the signal components of the deep layer portion and the surface layer portion in the biological optical measurement, methods of presenting the conditions and results to the user that have not conventionally taken into consideration are improved. Therefore, the user can perform analysis easily and accurately in the biological optical measurement.

In the embodiment described above, the deep portion component and the shallow portion component are separated from each other using the light received at two light receiving points of the short light receiving point and the long light receiving point having different SD distances. However, the number of light receiving points having different SD distances is not limited to 2. The number of light receiving points having different SD distances may be 3 or more. In addition, the short measurement data and the long measurement data may be measured by the probe arrangement having two or more irradiation points for one light receiving point, or each of the short measurement data and the long measurement data may be measured at a pair of one irradiation point and one light receiving point.

Although, in the embodiment described above, the control unit 140 of the biological optical measurement device 100 includes the data processing unit 400, the present invention is not limited thereto. The data processing unit 400 may also be provided in an information processing apparatus that is separated from the biological optical measurement device 100 and that can transmit and receive the data to and from the biological optical measurement device 100.

### REFERENCE SIGNS LIST

100: biological optical measurement device
110: light source unit
111: semiconductor laser
112: optical module
120: light receiving unit
121: photoelectric conversion element
122: lock-in amplifier
123: A/D converter
130: optical fiber
131: irradiation light optical fiber
132: light receiving optical fiber
140: control unit
141: input unit
142: display unit
143: storage unit
150: probe holder
151: probe
151a: irradiation point
151b: short light receiving point
151c: long light receiving point
190: object
300: light
301: light
302: light
400: data processing unit
410: analysis section
420: delay time determination section
430: display data generation section
511: long measurement data
512: short measurement data
521: first separated component
522: second separated component
531: deep portion component
532: shallow portion component
600: separation degree display screen
601: candidate delay time display region
602: separation degree display region
603: candidate delay time input region
604: input delay time separation degree display region
605: determination button
606: reception field
607: START button
608: progress bar
700: result display screen
701: waveform display field
702: percentage display field
703: information display field
711: deep blood flow waveform
712: shallow blood flow waveform
713: total blood volume

1. A biological optical measurement device, comprising:
one or more light source units that emit light to an irradiation point set in advance on an object;
one or more pairs of light receiving units that receive light emitted to the irradiation point and propagating through the object at a light receiving point set in advance on the object, that are disposed such that a distance between the irradiation point and the light receiving point for measuring short measurement data is shorter than a distance between the irradiation point and the light receiving point for measuring long measurement data, and that output at least one of the short measurement data and the long measurement data obtained from a received signal;
a data processing unit that performs processing on the short measurement data and the long measurement data; and
a display unit that displays the short measurement data and the long measurement data processed by the data processing unit and analysis data of the short measurement data and the long measurement data,
wherein the data processing unit includes:
an analysis section that extracts separated components by performing independent component analysis including a process of applying a delay time to at least one of a plurality of principal components obtained by an operation using at least one of the short measurement data and the long measurement data and separates the separated components into a deep portion component and a shallow portion component by reconstructing the separated components;
a delay time determination section that determines a delay time from candidate delay times indicated in the process of applying the delay time; and
a display data generation section that generates display data displayed on a display unit from a deep portion component and a shallow portion component obtained by applying the delay time.

2. The biological optical measurement device according to claim 1,
wherein the delay time determination section determines the candidate delay time by which a separation degree indicating a degree of separation of the shallow portion component and the deep portion component is maximized, among the candidate delay times, as an optimal delay time.

3. The biological optical measurement device according to claim 1,
wherein the delay time determination section further sets one or more predetermined times in a time range set in advance in a vicinity of a candidate delay time, by which a separation degree indicating a degree of separation of the shallow portion component and the deep portion component is maximized, to candidate delay times, calculates the separation degree for each of the candidate delay times, and determines the candidate delay time by which the separation degree is maximized as an optimal delay time.

4. The biological optical measurement device according to claim 1,
wherein the delay time determination section receives an input of at least one of the candidate delay times from a user, calculates a separation degree indicating a degree of separation of the shallow portion component and the deep portion component for the candidate delay time, and displays the separation degree on the display unit.

5. The biological optical measurement device according to claim 1,
wherein the delay time determination section receives a designation of the candidate delay time from a user, and determines the designated candidate delay time as the delay time.

6. The biological optical measurement device according to claim 1,
wherein the data processing unit further includes a display data generation section that generates display data displayed on the display unit from the deep portion component and the shallow portion component obtained by applying the delay time determined by the delay time determination section, and
the display data includes the delay time and at least one of waveforms of the separated components, a waveform of the deep portion component, and a waveform of the shallow portion component obtained by applying the determined delay time.

7. The biological optical measurement device according to claim 6,
wherein the short measurement data and the long measurement data are at least one of an oxygenated hemoglobin concentration change, a deoxygenated hemoglobin concentration change, and a total hemoglobin concentration change,
the analysis section calculates the separated components, the deep portion component, and the shallow portion component for each of the concentration changes obtained as the short measurement data and the long measurement data, and
the display data includes a percentage of the deep portion component for each of the concentration changes.

8. The biological optical measurement device according to claim 1,
wherein the data processing unit calculates a separation degree indicating a degree of separation of the shallow portion component and the deep portion component from a difference between an index of distribution of the shallow portion component and an index of distribution of the deep portion component, and displays the separation degree on the display unit.

9. The biological optical measurement device according to claim 8,
wherein the index of the distribution is obtained by dividing an average of root mean squares, which are calculated from time-series data of each component, of all measurement points by a standard deviation of all measurement points.

10. The biological optical measurement device according to claim 6,
wherein the display data generation section generates and displays the display data for each of the deep portion component and the shallow portion component obtained from the short measurement data and the long measurement data.

11. The biological optical measurement device according to claim 1,
wherein, when displaying a result of separation using the determined delay time, the display data generation section generates the display data so that waveforms, in which a cerebral blood flow signal and a skin blood flow signal before separation are mixed, a waveform based on a cerebral blood flow signal and a waveform based on a skin blood flow signal after separation, are displayed so as to be compared with each other.

12. The biological optical measurement device according to claim 1,
wherein the display data generated by the display data generation section includes a reception field to receive an instruction regarding whether to automatically determine or manually determine the delay time.

13. The biological optical measurement device according to claim 1,
wherein the delay time determination section determines the candidate delay time by which a mixing degree indicating a degree of mixing of the shallow portion component and the deep portion component is minimized, among the candidate delay times, as an optimal delay time.

14. The biological optical measurement device according to claim 1,
wherein the delay time determination section further sets one or more predetermined times in a time range set in advance in a vicinity of a candidate delay time, by which a mixing degree indicating a degree of mixing of the shallow portion component and the deep portion component is minimized, to candidate delay times, calculates the mixing degree for each of the candidate delay times, and determines the candidate delay time by which the mixing degree is minimized as an optimal delay time.

15. A signal separation method for a biological optical measurement device, comprising:
a step of emitting light to an irradiation point set in advance on an object using one or more light source units;
a step of receiving light emitted to the irradiation point and propagating through the object at a light receiving point set in advance on the object using one or more pairs of light receiving units, which are disposed on the object such that a distance between the irradiation point and the light receiving point for measuring short measurement data is shorter than a distance between the irradiation point and the light receiving point for measuring long measurement data, and of outputting at least one of the short measurement data and the long measurement data obtained from the received signal;
a data processing step of performing processing on the short measurement data and the long measurement data using a data processing unit; and
a step of displaying the short measurement data and the long measurement data processed by the data processing unit and analysis data of the short measurement data and the long measurement data on a display unit,
wherein the data processing step includes:
a step of extracting separated components by performing independent component analysis including a process of applying a delay time to at least one of a plurality of principal components obtained by an operation using at least one of the short measurement data and the long measurement data and separating the separated components into a deep portion component and a shallow portion component by reconstructing the separated components using an analysis section;
a step of determining a delay time from candidate delay times indicated in the process of applying the delay time using a delay time determination section; and
a step of generating display data displayed on the display unit from a deep portion component and a shallow portion component obtained by applying the delay time using a display data generation section.
